(19) United States
(12) Patent Application Publication
(10) Pub. No.: US 2025/0372218 A1
(43) Pub. Date: Dec. 4, 2025

(54) ARTIFICIAL INTELLIGENCE-ASSISTED ELECTRONIC HEALTH RECORD SYSTEMS AND METHODS

(71) Applicant: Carbon Health Technologies, Inc., Oakland, CA (US)

(72) Inventors: Murat Akbal, San Mateo, CA (US); Xin Sun, San Francisco, CA (US); Xinmiao Tan, Freemont, CA (US); Seak Meng Lay, San Francisco, CA (US); Haritha Atluri, Sacramento, CA (US); Neha Kumar, San Jose, CA (US); Caesar Djavaherian, Berkeley, CA (US); Eren Bali, Orange County, CA (US); Ivan Aslamov, Alameda, CA (US); Himanshu Agarwal, Behror (IN); Emre Açikgöz, Ankara (TR); Ekrem Küçük, Ankara (TR); Aditya Prakash Sai, Folsom, CA (US); Yuqi Shang, Irvine, CA (US)

(21) Appl. No.: 19/227,230

(22) Filed: Jun. 3, 2025

Related U.S. Application Data
(60) Provisional application No. 63/655,432, filed on Jun. 3, 2024.
(60) Provisional application No. 63/695,653, filed on Sep. 17, 2024.

(51) Int. Cl. G16H 10/60; G06F 40/186; G10L 25/66; G16H 40/20

(52) U.S. Cl. CPC G16H 10/60; G06F 40/186; G10L 25/66; G16H 40/20

(57) ABSTRACT

A vertically integrated native language-capable artificial intelligence assisted electronic health records system can include an electronic health records system equipped with a native artificial intelligence application. The system is configured to receive inputs from healthcare providers and retrieve relevant stored information to generate relevant outputs such as patient charts or administrative documents. The system can be run on various computing systems, including mobile tablets, mobile phones, or desktops.

### CROSS-REFERENCE TO RELATED APPLICATIONS

The present application claims the benefit of U.S. Provisional Patent Application No. 63/655,432, entitled “ARTIFICIAL INTELLIGENCE-ASSISTED ELECTRONIC HEALTH RECORD SYSTEMS AND METHODS”, filed Jun. 3, 2024, and U.S. Provisional Patent Application No. 63/695,653, entitled “ARTIFICIAL INTELLIGENCE-ASSISTED ELECTRONIC HEALTH RECORD SYSTEMS AND METHODS”, filed Sep. 17, 2024, which are herein incorporated by reference in their entirety.

### TECHNICAL FIELD

Embodiments relate generally to systems and methods for generating electronic health records, and more specifically to electronic health records systems and methods with native, vertically integrated artificial intelligence features.

### BACKGROUND

Healthcare providers compile charts of the medical data they collect from patients. Standard practice has become inputting this data as electronic health records (EHR) into various platforms that house these EHR systems. Traditional “charting” involves a healthcare provider manually entering collected information into the EHR system. In clinical settings, providers have to choose between connecting with their patients (e.g., speaking directly with and making eye contact) and recording notes, typically by typing. Further, healthcare systems around the world are overburdened, for a plethora of reasons, and cumbersome manual data collection restricts the number of patients that providers can reasonably see in a day. Traditional charting methods cut into time that could be spent seeing more patients or treating patients more comprehensively.

To combat these problems, providers that keep records manually may limit the number of details in the record they keep, be forced to use personal shorthand while meeting with patients, or miss recording details in order to focus on patient interaction. Incomplete records are a problem if a patient needs additional treatment in the future or sees a different healthcare provider, or if issues raised during an appointment are incorrectly, incompletely, or not at all recorded in the EHR.

In an effort to increase productivity and connection with patients, some healthcare providers have introduced artificial intelligence (AI) scribes into their charting practice. Typical AI scribes function as follows: the healthcare provider speaks their medical notes into the device that houses the AI scribe application, the scribe converts the audio data into written data, and that data is copied into the corresponding medical chart. While AI scribes remove the step of healthcare providers having to manually enter written data into electronic health records systems, they fall short of truly streamlining the process of collecting and processing patient's medical records and visit with a medical provider. Moreover, conventional scribes are add-on features to existing EHR systems, which can result in time lag, integration, cost, and other issues that affect performance.

### SUMMARY

Accordingly, there is a demonstrated need in electronic health record systems for a more efficient process that does not require manual data entry and generates relevant output materials from received input, such as patient charts or administrative forms. Such a system would aid healthcare providers in more efficiently and comprehensively meeting patients' needs.

Embodiments of the present disclosure relate to systems and methods that enable healthcare providers to generate comprehensive and accurate medical notes more efficiently, with vertical integration of a native artificial intelligence application directly into an EHR platform. Such embodiments comprise an EHR platform integrated with an artificial intelligence application that is configured to receive inputs from a healthcare provider, such as via speech and patient vitals or data, and the stored data in the EHR platform, such as patient-specific medical history, including care plan or in-take information provided by patient at the time appointment was scheduled, in order to generate outputs including but not limited to a complete healthcare chart, after-visit summary, diagnoses, and billing codes, among others.

In one embodiment, the system can be displayed on a mobile tablet or other computing device, such as a laptop computer, desktop computer, computing screen or terminal, smart phone, or other device. In such embodiments, the healthcare provider can use the system by interacting with a display, such as touching, tapping, or otherwise activating a “record” button, and then making verbal commentary. The system, with the assistance of the native artificial intelligence application, generates medical chart content based on the healthcare provider's commentary and the wealth of information already known by the system.

Hands-free charting starts with patient consent at the beginning of a visit. Once consent is obtained, with a simple press of a record button audio is captured and transcribed using a medical transcription system such as AWS Transcribe Medical. The transcript is then combined with other patient information such as demographics, vitals, lab results, and manually-added notes from the provider. Within minutes, a visit summary is auto-generated in the patient's chart, allowing a provider to review, adjust, and finalize it as the medical decision-maker.

The above summary is not intended to describe each illustrated embodiment or every implementation of the present disclosure. The detailed description that follows more particularly exemplifies these embodiments.

### BRIEF DESCRIPTION OF THE DRAWINGS

Subject matter hereof may be more completely understood in consideration of the following detailed description of various embodiments in connection with the accompanying figures, in which:

FIG. 1 is a diagram of a system for keeping electronic health records, having a native artificial intelligence, according to an embodiment.

FIG. 2 depicts an example approach for recording and sorting information, sorting and generating datasets to be fed to the AI agent according to potential embodiments.

FIG. 3 depicts an example approach for recording and sorting information, sorting and generating datasets to be fed to the AI agent for reporting according to potential embodiments

FIG. 4A depicts a system for keeping electronic health records as displayed on a mobile tablet, according to one embodiment.

FIG. 4B depicts a system for keeping electronic health records as displayed on a mobile phone, according to another embodiment.

FIG. 5 is a view of the system having an example health record displayed on a tablet screen.

While various embodiments are amenable to various modifications and alternative forms, specifics thereof, have been shown by way of example in the drawings and will be described in detail. It should be understood, however, that the intention is not to limit the claimed disclosures to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Current EHR systems rely on either manual data entry by healthcare providers or AI scribes that simply convert audio data into written data, copying the data into records without any further processing or filtering or dictation. Neither type of conventional system utilizes the range of information available in a patient EHR or information from in-take form or generates outputs that can aid the provider in treating the patient or streamlining the administrative process from a visit.

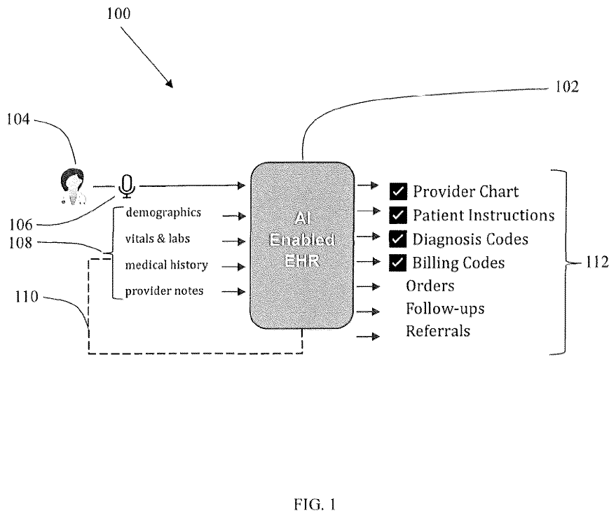

Accordingly, FIGS. 1-5 show non-limiting embodiments of a vertically integrated electronic health records system having a native AI application able to “learn” from input data and generate output in various forms.

Referring to FIG. 1, a system for hands-free health records comprising an electronic health records system 102 and having a native artificial intelligence application is depicted. The native AI application of the EHR system can be one of any suitable such applications, such as a generative pre-trained transformer (GPT), a large language model (LLM), or any other such system known to those who have skill in the art.

The EHR system 102 is configured to receive data input from a healthcare provider 104 (as well as a patient or others participating in a medical appointment, such the parent of a minor), via a microphone 106 (prior to recording, patient consent generally must be obtained) and retrieve stored data 108 from itself. This retrieved data 108 can include one or more of patient vitals, patient history, prescription records, laboratory orders or results, imaging results, photos, past provider notes, and any other patient-specific data that may be contained in an EHR or provided to an EHR as part of a patient care experience and history.

The EHR system 102 is further configured to generate one or more outputs 112. For example, outputs 112 can come in the form of compiled reports, including, but not limited to, provider charts, patient instructions, diagnosis codes, billing codes, prescription orders, laboratory or imaging orders, follow-ups, or referrals. The outputs 112 can also include raw data that is sent to other systems for further processing or instructions and in some instances may require sign-off or approval from a medical provider or clinician. The EHR system 102 also stores its outputs 112 in an EHR database to maintain a rich data collection. An internal prompt 110 sends the generated outputs 112 to retrievable storage in the EHR system 102.

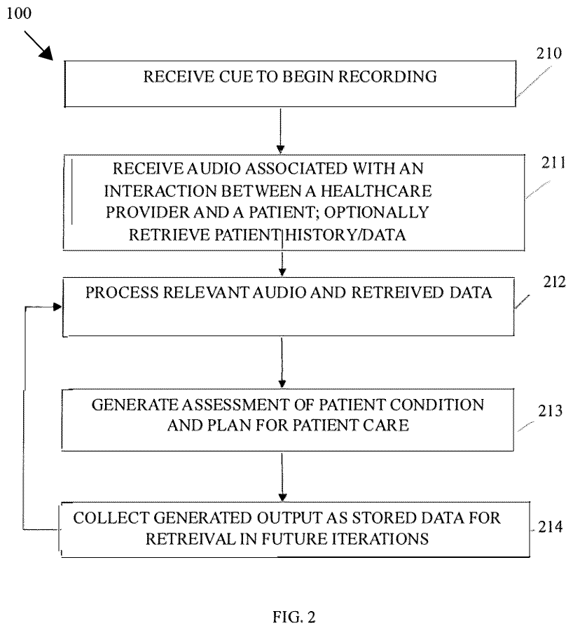

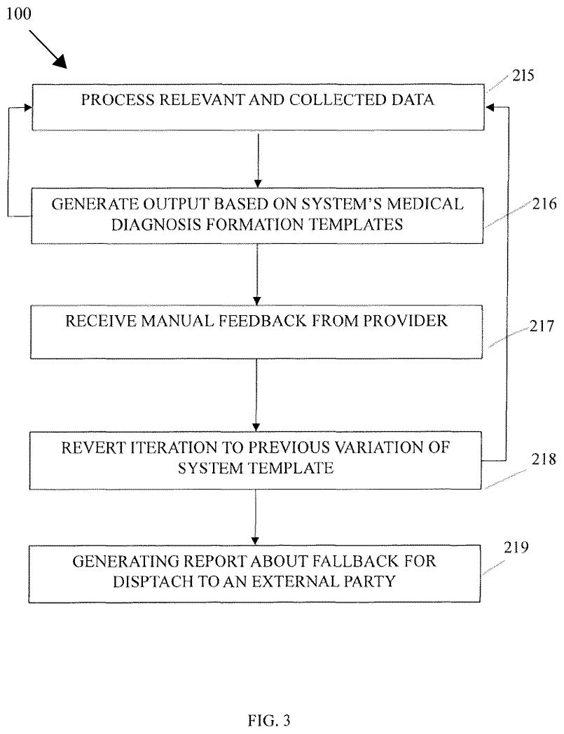

Referring to FIGS. 2 and 3, the EHR system 102 generally follows an industry standard subjective-objective-assessment-plan, or “SOAP,” process for medical decision-making formation. The user receives a cue to begin recording within the application 210. The SOAP process takes the subjective narrative of the patient and objective data collected during an appointment to make an assessment as to the condition of the patient and plan for treatment. The subjective inputs 211 may come from the audio input from the healthcare provider and patient at an appointment or from the rich data collection the EHR system 102 maintains on a particular patient. The objective inputs come from data collected from the patient, such as vital signs, lab results, imaging results, etc. 212.

The native AI application of the EHR system 102 then processes those inputs according to a system of templates and subtemplates 216. These templates and sub-templates can be customized according to a patient population, demographic, provider preference or requirement, or some other clinical characteristic(s). Accordingly, these prompts can be custom engineered to guide the EHR system 102 to obtain (or identify potentially relevant) information for a particular application, user, site, or setting. This information then can be used to provide clinically relevant generated outputs 112, such as an assessment, potential diagnosis or diagnoses, or a proposed plan of treatment, follow up, or further evaluation 213. Other generated outputs may be administrative in nature, such as billing codes.

The EHR system 102 benefits from a feedback loop, enabling the system 100 to quickly generate improved iterations of its input processing and output 216 generation. Clinicians and other providers can provide feedback on the output 112 generated by the system 100, creating a quantified feedback loop 214. This feedback loop “teaches” the native AI of the EHR system 102 which parts of the medical decision making formation in the generated outputs 112 are correct/incorrect, relevant/irrelevant, or otherwise worth review or updating. Further, human scribes, clinicians, medical directors, and others who interact with the system 100 can provide more feedback 217, further strengthening the quantified feedback loop.

This quantified feedback loop can also have precautionary variant fallback logic. For example, referring to FIG. 3, in an instance where the EHR system 102 receives feedback from a provider that a generated output is beyond some predetermined error range or where the system does not or cannot identify a potential output (where such an output is either validated to some level by the system 100), the system 100 will employ variant fallback logic to return 218 to the last successful iteration of the native AI application EHR system 215. This inhibits the native AI application from carrying a significant error into further iterations. The predetermined error range may be based on the total number of errors found by the provider in a particular generated output, the magnitude of the error, a similarity of similar input or output, or any other suitable prompt. The system 100 can also generate and send a report 219 to an external party about the system 100 triggering fallback logic, to prompt investigation or manual intervention or review, for example.

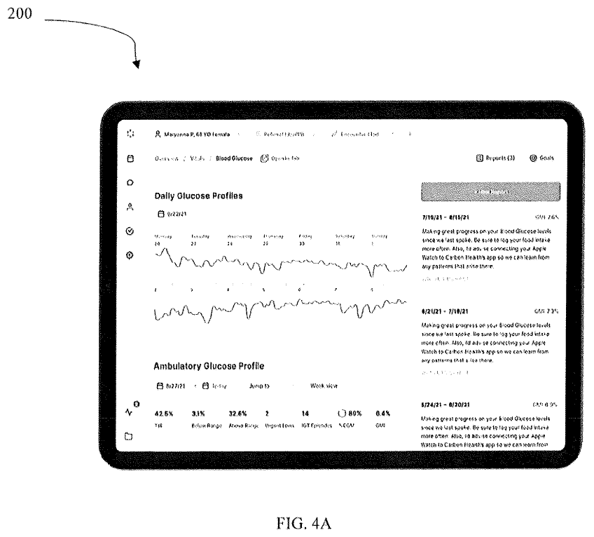

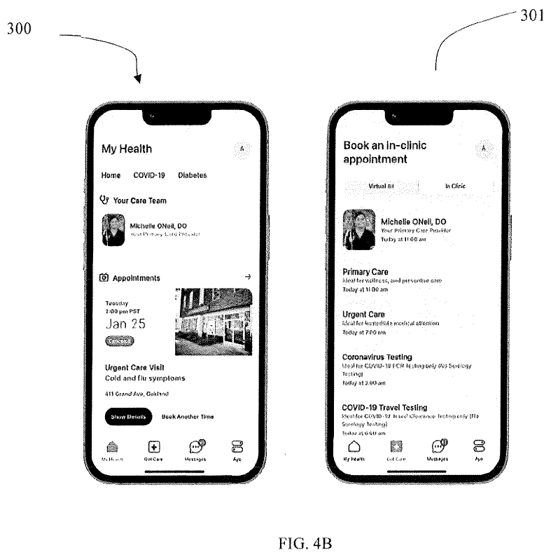

System 100 can be compatible with various computing systems, including, but not limited to, mobile tablets, mobile phones, and desktops, including medical terminal interfaces. The user interface displayed on the system may vary depending on the type of computing. Referring to the examples of FIGS. 4A system 200 and 4B, the user interface of system 300 are adapted for a mobile tablet and a mobile phone, respectively. Screen 301 illustrates the steps for booking an in-clinic appointment.

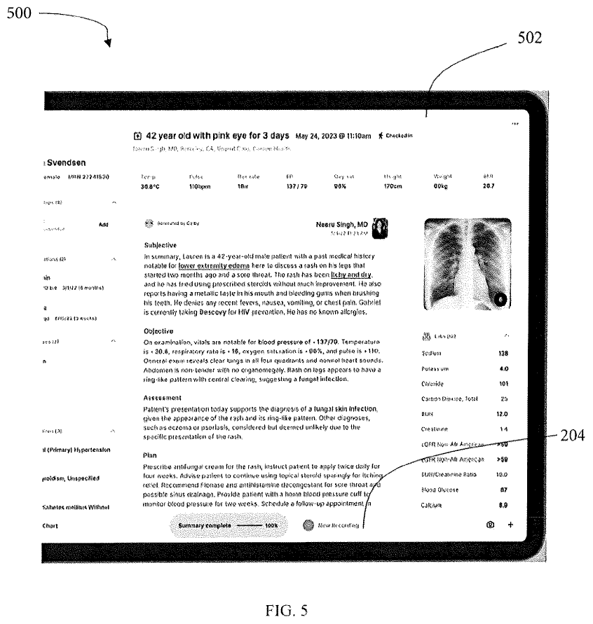

FIG. 5 demonstrates an exemplary display 502 that may be generated by the EHR system for viewing by the healthcare provider.

In some embodiments, the EHR system 102 further comprises virtual assistant technology, enabling background actions to be performed while the EHR system is still collecting data from the provider and patient based on detected “wake” or “trigger” words or phrases. Virtual assistant technology is dormant until a specific prompt is spoken, enabling it to be ready at any time to assist. In one embodiment, the hands-free voice interaction is enabled by prompting a virtual assistant, called “CARBY,” to start an action. For example, during an appointment, a healthcare provider may prompt “CARBY” by saying “CARBY, fill a prescription for . . . ” and the EHR system 102 will complete a prescription script for final approval by the provider, while the provider continues the appointment. “CARBY” may also be used to record relevant vitals, such as by saying, “CARBY, the blood pressure is 120/70.” This deep linking can enable a provider to continue to focus on patient interaction without needing to pause to initiate other tasks, such as prescription orders, lab orders, imaging requests, referrals, and others, which can be carried out, at least partially, in the background and hands-free.

Advantageously, system 100 is configured to fit into common healthcare provider workflows. Using FIG. 5 which is a tablet application 500 as an example, upon receiving a patient and obtaining consent from patient to record the appointment, the healthcare provider simply presses a record button 204 on the display 502, prompts the virtual assistant, or otherwise initiates recording, and the EHR system 102 begins capturing audio and transcribes the recording using a transcription program. Though video recording may raise additional privacy concerns and therefore may not be preferred by clinicians or patients, system 100 can also accept video, photographic, or other imaging data as input. The provider may further manually include images (e.g., photographs, X-rays, scans) of a patient's condition or injuries for inclusion in the chart.

In embodiments, the transcription programs can be any suitable program, such as an AI scribe for medical applications. The EHR system then processes the transcript and combines it with other patient data stored on the EHR system 102, including current and past patient demographics, vitals, lab results, and manually added notes from the provider. The EHR system 102 can then generate an output 112 for review by the healthcare provider or to comply with administrative procedures.

In an embodiment, the invention is pioneering AI-assisted orders, including but not limited to labs and prescriptions. The recommendations are generated during a provider-patient conversation. The transcript of the conversation thus far, along with metadata about the patient and appointment, are fed into a multi-step process diagrammed below:

The AI system contains three components as illustrated in Table 1 above. The first component determines what order types are necessary (for example, “labs” or “prescription”). The second component retrieves particulars of the order type returned by the first component. This includes internal identifiers for the exact order that needs to be placed along with human readable helper text. Finally, a post-processing layer validates the AI generated outputs against historical data.

In an embodiment, the Charge Navigator marks a significant enhancement in the realm of medical billing and coding, specifically targeting the challenge of accurately coding dual nature appointments that require both a wellness code and an E&M code, or a combination thereof, including procedure codes. This tool is an expansion of the initial MDM-based coding calculator, now accommodating the complexity of appointments needing multiple coding categories due to their dual nature. By integrating an initial question within the MDM coding calculator regarding the appointment type—pre-populated based on appointment chief complaint (CC) and specialty but adjustable by the provider—this tool ensures precise coding by preventing the addition of E&M codes via the charge navigator for specific visit types.

Fields that inform the E&M code calculation are populated via AI. Candidate values for each field, a description of each candidate value, and descriptions of the fields overall are dynamically injected into the prompt. The dynamic nature ensures forward compatibility with evolving coding guidelines.

This enhancement not only aims at improving coding accuracy and reducing claim rejections but also at simplifying the billing process for providers, ultimately leading to more efficient healthcare delivery and financial operations. By accurately capturing the complexity of dual-nature appointments, the tool aligns with broader organizational goals of enhancing billing accuracy, provider compliance, and overall operational efficiency. The Table 2 below illustrates these enhancements.

Thus, embodiments disclosed herein related to vertically integrated, native language-capable EHR systems and methods with smart, hands-free charting and task provision. Engineering prompts can train the AI features of the system, both initially and iteratively on an ongoing basis, while the system also continues to learn from any edits, corrections, adjustments, or other inputs made thereto by clinicians or other providers. This engineering and iterative approach provides opportunities for new prompts to be added, existing prompts to improved, and unhelpful or unnecessary prompts to be eliminated, making the system smarter and more accurate. Furthermore, outputs also can be engineered or customized according to provider preference, style, care setting, or some other factor. Embodiments therefore can save time, make care appointments more patient-centric, reduce opportunities for errors or missed tasks, improve the patient and provider experience, and enable everyone involved in a patient's care to focus on real care rather than documentation.

The disclosure may be embodied in other specific forms without departing from the essential attributes; therefore, the illustrated embodiments should be considered illustrative and not restrictive in all respects. The claims provided herein are to ensure adequacy of the present application for establishing foreign priority and for no other purpose.

Various embodiments of systems, devices, and methods have been described herein. These embodiments are given only be way of example and are not intended to limit the scope of the claimed disclosures. It should be appreciated, moreover, that the various features of the embodiments that have been described may be combined in various ways to produce numerous additional embodiments. Moreover, while various material, dimensions, shapes, configurations, locations, etc. have been described for use with disclosed embodiments, others besides those disclosed may be utilized without exceeding the scope of the claimed disclosures.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Though embodiments discussed herein may relate to example clinical settings, these settings are not limiting and others are possible, including dental, emergency, urgent care, surgical, triage, optometric, and any other setting in which, generally speaking, a clinician or provider interacts with one or more patients and a note and or record-keeping system that is or is similar to an EHR system.

Any incorporation of reference of documents above is further limited such that no claims included in the documents are incorporated by reference herein. Any incorporation by reference of documents above is yet further limited such that any definitions provided in the documents are not incorporated by reference herein unless expressly included herein.

What is claimed is:

1. A system for electronic health record (EHR) generation and collection comprising;
a custom engineered application with a native artificial intelligence system that processes a patient's health data and inputs according to a system of templates and sub-templates, wherein the templates and sub-templates can be customized according to a patient population, demographic, provider preference or requirement, or some other clinical characteristic; and
wherein the native artificial intelligence system guides the EHR system to obtain relevant information for a particular application, user, site, or setting to provide clinically relevant generated outputs such as an assessment, potential diagnosis or diagnoses, or a proposed plan of treatment, follow up, or further evaluation; and
wherein the EHR system includes a feedback loop, enabling the system to quickly generate improved iterations of its input processing and output generation based on input from clinicians and other, wherein said feedback loop “teaches” the native artificial intelligence system of the EHR system which parts of the medical decision making formation in the generated outputs are correct/incorrect, relevant/irrelevant, or otherwise worth review or updating.

2. A method for generating and collecting electronic health records, including a native artificial intelligence machine, the method comprising:
accepting, by a computing device, a selection of a demographic characteristic of users using a first version of a product;
extracting, from a set of historical session logs of the users using the first version of the product, a subset of session logs of users having the demographic characteristic, wherein the subset excludes users not having the demographic characteristic;
training an Artificial Intelligence (AI) agent to use the first version of the product to perform a first set of tasks, wherein training the AI agent comprises applying one or more machine learning models to the subset of session logs, wherein applying the one or more machine learning models comprises at least one of:
applying a pattern recognition model or a classification model to recognize normal or abnormal patterns of user behavior;
applying a regression model to identify causal factors for one or more error messages received while using the first version of the product; or
applying a decisioning model to identify actions suited to achieving particular tasks based on available options while using the first version of the product;
instructing the AI agent to perform, using a second version of the product, at least one of the first set of tasks or a second set of tasks, the second version of the product modified from the first version of the product to include a new or modified feature not in the first version of the product; and
generating, by the computing device, a report of the AI agent using the first version of the product or the AI agent using the second version of the product.

3. A method implemented by a machine learning platform, the method comprising:
receiving a selection of audio associated with an interaction between a healthcare worker and a patient related to patient issues and data;
applying machine learning techniques to the determine relevant audio associated with an interaction between a healthcare worker and a patient related to patient issues and data;
generating an assessment of the patient condition and a plan for patient care;
collecting generated assessments and store for future iterations;
generating a feedback loop to introduce the collected generated assessments for inclusion in the generating assessment of patient care step to quickly generate improved iterations of its input processing and output generation based on input from clinicians and other sources, said feedback loop informing which parts of the patient care plan are correct/incorrect, relevant/irrelevant, or otherwise worth review or updating; and
updating the plan of patient care.
